(19) United States
(12) Patent Application Publication
(10) Pub. No.: US 2007/0117834 A1
(43) Pub. Date: May 24, 2007

(54) Methods and compositions for treating Huntington's disease

(76) Inventor: David Hung, San Francisco, CA (US)

Correspondence Address: MORRISON & FOERSTER LLP, 755 PAGE MILL RD, PALO ALTO, CA, 94304-1018, US

(21) Appl. No.: 11/543,341

(22) Filed: Oct. 4, 2006

Related U.S. Application Data
(60) Provisional application No. 60/723,403, filed on Oct. 4, 2005.

(51) Int. Cl. A61K 31/4745

(52) U.S. Cl. 514/291

(57) ABSTRACT

The invention provides method for treating Huntington's disease, slowing the onset and/or development and/or progression of Huntington's disease or preventing the development of Huntington's disease using hydrogenated pyrido[4,3-b]indoles, including dimebon.

### CROSS-REFERENCE TO RELATED APPLICATIONS

This application claims priority to U.S. Provisional Patent Application No. 60/723,403, filed Oct. 4, 2005, which is incorporated herein by reference in its entirety.

### STATEMENT OF RIGHTS TO INVENTIONS MADE UNDER FEDERALLY SPONSORED RESEARCH

Not applicable.

### TECHNICAL FIELD

The present invention is related to the use of hydrogenated pyrido[4,3-b]indoles or pharmaceutically acceptable salts thereof in the area of medicine, which may be used as agents for treating, preventing or delaying the onset and/or development of Huntington's disease when they are prepared as pharmacological compositions.

### BACKGROUND OF THE INVENTION

Huntington's disease is a fatal neurological disorder characterized clinically by symptoms such as involuntary movements, cognition impairment or loss of cognitive function and a wide spectrum of behavioral disorders. Common motor symptoms associated with Huntington's disease include chorea (involuntary writhing and spasming), clumsiness, and progressive loss of the abilities to walk, speak (e.g., exhibiting slurred speech) and swallow. Other symptomatic aspects of Huntington's disease can include cognitive symptoms such as loss of intellectual speed, attention and short-term memory and/or behavioral symptoms that can span the range of changes in personality, depression, irritability, emotional outbursts and apathy. It is estimated that 30,000 patients currently suffer from Huntington's disease in the US alone with estimates of its prevalence at 1 in every 10,000 persons. The worldwide incidence of Huntington's disease is much larger. Furthermore, at least an additional 150,000 people in the US alone are genetically at risk of being a carrier of the Huntington's disease gene which is responsible for the clinical syndrome of Huntington's disease but whose effects unfortunately do not generally become clinically apparent until the fourth or fifth decade of life.

In patients with Huntington's disease, death usually occurs approximately 10-20 years after the onset of symptoms, making this disease not only a devastating illness but also a protracted illness. Hence, patients suffering from Huntington's disease are in great need of a medicament that can treat the disease and/or reduce the behavioral and/or motor and/or cognitive symptoms associated with the disease.

Huntington's disease is inherited through a mutated or abnormal gene which codes for an abnormal protein called the mutant huntingtin protein. Huntington's disease is known to be caused by a specific genetic mutation, which results in degeneration of neurons in many different regions of the brain. This degeneration is particularly focused in neurons located in the basal ganglia, structures deep within the brain that control many important functions including coordinating movement, and also in neurons on the outer surface of the brain or cortex, which controls thought, perception and memory.

Currently there is no cure for Huntington's disease, and there are no therapies which slow the progression of the devastating disease or delay its onset and/or development. There are no FDA approved treatments for Huntington's disease in the US, and the disease is invariably fatal. Everyone who carries at least one copy of the Huntington's disease mutation and lives long enough will develop the disease. Symptoms generally begin between the ages of 30 and 45, but have been reported to appear as early as two years of age.

Physicians attempt to control the symptoms of patients suffering from Huntington's disease, and are known to prescribe antipsychotic drugs such as haloperidol to Huntington's disease patients to try to control hallucinations, delusions and violent outbursts; or antidepressants for depression; or tranquilizers for anxiety control; or lithium for pathological excitement or mood swings; or memantine, amantadine or cholinesterase inhibitors to try to control the movement disorders.

Known compounds of the class of tetra- and hexahydro-1H-pyrido[4,3-b]indole derivatives manifest a broad spectrum of biological activity. In the series of 2,3,4,5-tetrahydro-1H-pyrido[4,3-b]indoles the following types of activity have been found: antihistamine activity (DE 1,813,229, filed Dec. 6, 1968; DE 1,952,800, filed Oct. 20, 1969), central depressive and anti-inflammatory activity (U.S. Pat. No. 3,718,657, filed Dec. 3, 1970), neuroleptic activity (Herbert C. A., Plattner S. S., Welch W. M.—Mol. Pharm. 1980, v. 17, N 1, p. 38-42) and others. 2,3,4,4a,5,9b-hexahydro-1H-pyrido[4,3-b]indole derivatives show psychotropic (Welch W. M., Harbert C. A., Weissman A., Koe B. K. J. Med. Chem., 1986, vol. 29, No. 10, p. 2093-2099), antiaggressive, antiarrhythmic and other types of activity.

Several drugs, such as diazoline (mebhydroline), dimebon, dorastine, carbidine (dicarbine), stobadine and gevotroline, based on tetra- or hexahydro-1H-pyrido[4,3-b]indole derivatives are known to have been manufactured. Diazoline (2-methyl-5-benzyl-2,3,4,5-tetrahydro-1H-pyrido[4,3-b]indole dihydrochloride) (Klyuev M. A., Drugs, used in “Medical Pract.”, USSR, Moscow, “Meditzina” Publishers, 1991, p. 512) and dimebon (2,8-dimethyl-5-(2-(6-methyl-3-pyridyl)ethyl-2,3,4,5-tetrahydro-1H-pyrido[4,3-b]indole dihydrochloride) (M. D. Mashkovsky, “Medicinal Drugs” in 2 vol. Vol. 1-12th Edition, Moscow, “Meditzina” Publishers, 1993, p. 383) as well as dorastine (2-methyl-8-chloro-5-[2-(6-methyl-3-pyridyl)ethyl]-2,3,4,5-tetrahydro-1H-pyrido[4,3-b]indole dihydro chloride) (USAN and USP dictionary of drugs names (United States Adopted Names, 1961-1988, current US Pharmacopoeia and National Formula for Drugs and other nonproprietary drug names), 1989, 26th Edition., p. 196) are known as antihistamine drugs; carbidine (dicarbine) (cis(±)-2,8-dimethyl-2,3,4,4a,5,9b-hexahydro-1H-pyrido[4,3-b]indole dihydrochloride) is a neuroleptic agent having an antidepressive effect (L. N. Yakhontov, R. G. Glushkov, Synthetic Drugs, ed. by A. G. Natradze, Moscow, “Meditzina” Publishers, 1983, p. 234-237), and its (−)isomer, stobadine, is known as an antiarrythmic agent (Kitlova M., Gibela P., Drimal J., Bratisl. Lek. Listy, 1985, vol. 84, No. 5, p. 542-549); gevotroline 8-fluoro-2-(3-(3-pyridyl)propyl)-2,3,4,5-tetrahydro-1H-pyrido[4,3-b]indole dihydrochloride is an antipsychotic and anxiolytic agent (Abou-Gharbi M., Patel U. R., Webb M. B., Moyer J. A., Ardnee T. H., J. Med. Chem., 1987, vol. 30, p. 1818-1823). Dimebon has been used in medicine as an antiallergic agent (Inventor's Certificate No. 1138164, IP Class A61K 31/47,5, C07 D 209/52, published on Feb. 7, 1985) in Russia for over 20 years.

As described in U.S. Pat. No. 6,187,785, hydrogenated pyrido[4,3-b]indole derivatives, such as dimebon, have NMDA antagonist properties, which makes them useful for treating neurodegenerative diseases, such as Alzheimer's disease. See also U.S. Pat. No. 7,071,206. As described in WO 2005/055951, hydrogenated pyrido[4,3-b]indole derivatives, such as dimebon, are also useful as human or veterinary geroprotectors e.g., by delaying the onset and/or development of an age-associated or related manifestation and/or pathology or condition, including disturbance in skin-hair integument, vision disturbance and weight loss.

There is significant interest in and need for medications and agents for the treatment, prevention, slowing the progression and/or delaying the onset and/or development of Huntington's disease.

### BRIEF SUMMARY OF THE INVENTION

The present invention provides methods and compositions for treating, preventing, slowing the progression and/or delaying the onset and/or development of Huntington's disease comprising administering to an individual an effective amount of a hydrogenated pyrido[4,3-b]indole or pharmaceutically acceptable salt thereof, such as any acid or base salt thereof. The hydrogenated pyrido[4,3-b]indole can be a tetrahydro pyrido[4,3-b]indole or pharmaceutically acceptable salt thereof. The hydrogenated pyrido[4,3-b]indole can be a hexahydro pyrido[4,3-b]indole or pharmaceutically acceptable salt thereof. These compounds may be administered in the form of a pharmacological (this term is used interchangeably herein with “pharmaceutical”) composition which contains one or more pharmaceutically acceptable excipients. In one variation, the compound is dimebon.

Use of the compounds can be for the treatment, prevention, slowing the progression and/or delaying the onset and/or development of Huntington's disease that entail giving to an individual a pharmacological medication which contains an effective amount of a substance described herein, such as a compound described by Formula A, (1), B or (2). The compounds can be administered in an effective dose. The compounds can be administered in any dose disclosed herein, such as in a dose of about 0.1 to about 10 mg/kg of the body weight. The compounds can be administered in any dosing regimen and/or form disclosed herein, such as dosing at least once a day or dosing via an extended release dosing form.

The invention also provides kits for the methods described herein, such as kits comprising any of the compounds or pharmaceutical compositions disclosed herein.

### BRIEF DESCRIPTION OF THE DRAWINGS

FIG. 1 illustrates the minimal toxicity of dimebon in Drosophila (fruit fly).

FIG. 2 illustrates dimebon's ability to suppress degeneration of photoreceptor neurons in a Drosophila (fruit fly) model at concentrations ranging from 10 μM to 1 mM.

FIG. 3 illustrates dimebon's ability to suppress degeneration of photoreceptor neurons in a Drosophila (fruit fly) model at concentrations ranging from 100 μM to 1 mM. The assay showed significant rescue for the 100 μM dosage, as indicated by an asterisks * P<0.05 by Dunnett's test.

FIG. 4 illustrates dimebon's tendency to suppress degeneration of photoreceptor neurons in a Drosophila (fruit fly) model at concentrations ranging from 1 μM to 30 μM. The photoreceptor rescue of the tested concentrations of dimebon suggested a modest tendency for improved photoreceptor numbers, although differences were not statistically demonstrable.

FIG. 5 illustrates the climbing behavior (measured over 10 seconds) of Drosophila (fruit fly) reared on 10, 100 or 1,000 μM dimebon. From an initial number of 20 animals on each concentration at day 0, the numbers surviving to be tested at day 7 were: control: 12; 10 μM: 8; 100 μM: 12; 1 mM: 8. The highest and lowest performing animal from each set was discarded. Distances climbed in 10 seconds are given in arbitrary units from graduated cylinders. P<0.05 at 1 mM is indicated by the single asterisks *.

FIG. 6 shows scatter plots of raw climbing data (measured over 30 seconds) of Drosophila (fruit fly) reared on 10, 100 or 1,000 μM dimebon for two independent trials (A and B). Each point represents the average of three trials of a single fly. The variance is evident from the plots. D0 refers to Day 0 and D7 refers to Day 7 with no drug (ND) and dimebon at the concentrations indicated. Analysis indicated a statistical difference between D7ND and 1 mM in panel A and between D0 and D7ND in panel B.

### DETAILED DESCRIPTION OF THE INVENTION

It has been discovered that dimebon, a representative member of a class of compounds disclosed herein, had strikingly positive results in the art-accepted Drosophila model of Huntington's disease, and exhibited enhanced protective effects when compared to a control. The Drosophila fruit fly is considered an excellent choice for modeling neurodegenerative diseases because it contains a fully functional nervous system with an architecture that separates specialized functions such as vision, smell, learning and memory in a manner not unlike that of mammalian nervous systems. Furthermore, the compound eye of the fruit fly is made up of hundreds of repeating constellations of specialized neurons which can be directly visualized through a microscope and upon which the ability of potential neuroprotective drugs to directly block neuronal cell death can easily be assessed. Finally, among human genes known to be associated with disease, approximately 75% have a Drosophila fruit fly counterpart. Further discussion of the suitability of this model and its predictive value is found in the examples, such as in Example 2. Thus, dimebon and the compounds disclosed herein are believed to be a new class of compounds useful for the treatment, prevention, slowing the progression and/or delaying the onset and/or development of Huntington's disease.

For use herein, unless clearly indicated otherwise, use of the terms “a”, “an” and the like refers to one or more. It is also understood and clearly conveyed by this disclosure that reference to “the compound” or “a compound” includes and refers to any compound or pharmaceutically acceptable salt or other form thereof as described herein, such as the compound dimebon.

For use herein, unless clearly indicated otherwise, “an individual” as used herein intends an animal, such as a mammal, including but not limited to a human. In one aspect of the invention, the individual is a human who manifests one or more symptoms of Huntington's disease, such as involuntary movement and/or cognition impairment and/or behavioral symptoms. In one embodiment, the individual is a human who manifests one or more symptoms of Huntington's disease selected from any one or more of chorea, clumsiness, slurred speech, loss in intellectual speed, loss in ability to pay attention, loss in short-term memory, changes in personality, depression, irritability, emotional outbursts, and apathy. In one embodiment, the individual is a human who has been diagnosed with Huntington's disease. In one embodiment, the individual is a human who is considered to be at risk for developing Huntington's disease, for example, to an individual who has a mutated gene which codes for the mutant huntingtin protein or whose family history indicates that one or more family members has had Huntington's disease. In one embodiment, the individual is a human who is genetically predisposed to developing Huntington's disease. In one embodiment, the individual is a human who has a mutated or abnormal gene that codes for the mutant huntingtin protein. In one embodiment, the individual is a human who expresses a mutant huntingtin protein. In one variation, the individual is a human who has not been diagnosed with and/or is not considered at risk for developing Alzheimer's disease. In one variation, the individual is a human who has not been diagnosed with and/or is not considered at risk for developing Alzheimer's disease but who has or is considered at risk for developing Huntington's disease. In one variation, the individual is a human who does not have a cognition impairment associated with aging or does not have a non-life threatening condition associated with the aging process (such as loss of sight (cataract), deterioration of the dermatohairy integument (alopecia) or an age-associated decrease in weight due to the death of muscular and fatty cells) or a combination thereof. In one variation, the individual is a human who does not have a cognition impairment associated with aging or does not have a non-life threatening condition associated with the aging process (such as loss of sight (cataract), deterioration of the dermatohairy integument (alopecia) or an age-associated decrease in weight due to the death of muscular and fatty cells) or a combination thereof but who has or is considered at risk for developing Huntington's disease.

The term “effective amount” intends such amount of a compound described by the Formula (1) or by Formula (2) or any compound described herein, such as any compound described by the Formula (A) or (B), which in combination with its parameters of efficacy and toxicity, as well as based on the knowledge of the practicing specialist should be effective in a given therapeutic form. As is understood in the art, an effective amount may be in one or more doses. As is understood in the clinical context, an effective dosage of a drug, compound or pharmaceutical composition may or may not be achieved in conjunction with another drug, compound or pharmaceutical composition. Thus, an effective amount may be considered in the context of administering one or more therapeutic agents, and a single agent may be considered to be given in an effective amount if, in conjunction with one or more other agents, a desirable or beneficial result may be or is achieved.

### Compounds for Use in the Methods, Formulations, Kits and Inventions Disclosed Herein

When reference to organic residues or moieties having a specific number of carbons is made, unless clearly stated otherwise, it intends all geometric isomers thereof. For example, “butyl” includes n-butyl, sec-butyl, isobutyl and t-butyl; “propyl” includes n-propyl and isopropyl.

The term “alkyl” intends and includes linear, branched or cyclic hydrocarbon structures and combinations thereof. Preferred alkyl groups are those having 20 carbon atoms (C20) or fewer. More preferred alkyl groups are those having fewer than 15 or fewer than 10 or fewer than 8 carbon atoms.

The term “lower alkyl” refers to alkyl groups of from 1 to 5 carbon atoms. Examples of lower alkyl groups include methyl, ethyl, propyl, isopropyl, butyl, sec- and t-butyl and the like. Lower alkyl is a subset of alkyl.

The term “aryl” refers to an unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxain-3(4H)-one-7-yl), and the like. Preferred aryls includes phenyl and naphthyl.

The term “heteroaryl” refers to an aromatic carbocyclic group of from 2 to 10 carbon atoms and 1 to 4 heteroatoms selected from oxygen, nitrogen and sulfur within the ring. Such heteroaryl groups can have a single ring (e.g., pyridyl or furyl) or multiple condensed rings (e.g., indolizinyl or benzothienyl). Examples of heteroaryl residues include, e.g., imidazolyl, pyridinyl, indolyl, thiopheneyl, thiazolyl, furanyl, benzimidazolyl, quinolinyl, isoquinolinyl, pyrimidinyl, pyrazinyl, tetrazolyl and pyrazolyl.

The term “aralkyl” refers to a residue in which an aryl moiety is attached to the parent structure via an alkyl residue. Examples are benzyl, phenethyl and the like.

The term “heteroaralkyl” refers to a residue in which a heteroaryl moiety is attached to the parent structure via an alkyl residue. Examples include furanylmethyl, pyridinylmethyl, pyrimidinylethyl and the like.

The term “substituted heteroaralkyl” refers to heteroaralkyl groups which are substituted with from 1 to 3 substituents, such as residues selected from the group consisting of hydroxy, alkyl, alkoxy, alkenyl, alkynyl, amino, aryl, carboxyl, halo, nitro and amino. Substituted aralkyl refers to aralkyl groups which are substituted with from 1 to 3 substituents, such as residues selected from the group consisting of hydroxy, alkyl, alkoxy, alkenyl, alkynyl, amino, aryl, carboxyl, halo, nitro and amino.

The term “halo” or “halogen” refers to fluoro, chloro, bromo and iodo.

Compounds for use in the systems, methods and kits described herein are hydrogenated pyrido[4,3-b]indoles or pharmaceutically acceptable salts thereof, such as an acid or base salt thereof. A hydrogenated pyrido[4,3-b]indole can be a tetrahydro pyrido[4,3-b]indole or pharmaceutically acceptable salt thereof. The hydrogenated pyrido[4,3-b]indole can also be a hexahydro pyrido[4,3-b]indole or pharmaceutically acceptable salt thereof. The hydrogenated pyrido[4,3-b]indole compounds can be substituted with 1 to 3 substituents, although unsubstituted hydrogenated pyrido[4,3-b]indole compounds or hydrogenated pyrido[4,3-b]indole compounds with more than 3 substituents are also contemplated. Suitable substituents include but are not limited to alkyl, lower alkyl, aralkyl, heteroaralkyl, substituted heteroaralkyl, substituted aralkyl and halo.

Particular hydrogenated pyrido[4,3-b]indoles are exemplified by the Formulae A and B: 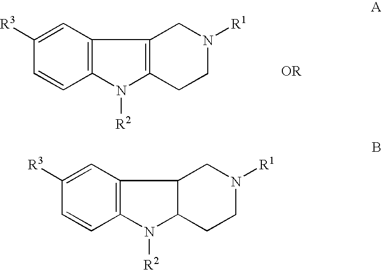
where R¹ is selected from the group consisting of alkyl, lower alkyl and aralkyl; R² is selected from the group consisting of hydrogen, aralkyl and substituted heteroaralkyl; and R³ is selected from the group consisting of hydrogen, alkyl, lower alkyl and halo.

In one variation, R¹ is alkyl, such as an alkyl selected from the group consisting of C₁-C₁₅alkyl, C₁₀-C₁₅alkyl, C₁-C₁₀alkyl, C₂-C₁₅alkyl, C₂-C₁₀alkyl, C₂-C₈alkyl, C₄-C₈alkyl, C₆-C₈alkyl, C₆-C₁₅alkyl, C₁₅-C₂₀alkyl, C₁-C₈alkyl and C₁-C₆alkyl. In one variation, R¹ is aralkyl. In one variation, R¹ is lower alkyl, such as a lower alkyl selected from the group consisting of C₁-C₂alkyl, C₁-C₄alkyl, C₂-C₄ alkyl, C₁-C₅ alkyl, C₁-C₃alkyl and C₂-C₅alkyl.

In one variation, R¹ is a straight chain alkyl group. In one variation, R¹ is a branched alkyl group. In one variation, R¹ is a cyclic alkyl group.

In one variation, R¹ is methyl. In one variation, R¹ is ethyl. In one variation, R¹ is methyl or ethyl. In one variation, R¹ is methyl or an aralkyl group such as benzyl. In one variation, R¹ is ethyl or an aralkyl group such as benzyl.

In one variation, R¹ is an aralkyl group. In one variation, R¹ is an aralkyl group where any one of the alkyl or lower alkyl substituents listed in the preceding paragraphs is further substituted with an aryl group (e.g., Ar-C₁-C₆alkyl, Ar-C₁-C₃alkyl or Ar-C₁-C₁₅alkyl). In one variation, R¹ is an aralkyl group where any one of the alkyl or lower alkyl substituents listed in the preceding paragraphs is substituted with a single ring aryl residue. In one variation, R¹ is an aralkyl group where any one of the alkyl or lower alkyl substituents listed in the preceding paragraphs is further substituted with a phenyl group (e.g., Ph-C₁-C₆Alkyl, Ph-C₁-C₃Alkyl or Ph-C₁-C₁₅alkyl). In one variation, R¹ is benzyl.

All of the variations for R¹ are intended and hereby clearly described to be combined with any of the variations stated below for R² and R³ the same as if each and every combination of R¹, R² and R³ were specifically and individually listed.

In one variation, R² is H. In one variation, R² is an aralkyl group. In one variation, R² is a substituted heteroaralkyl group. In one variation, R² is hydrogen or an aralkyl group. In one variation, R² is hydrogen or a substituted heteroaralkyl group. In one variation, R² is an aralkyl group or a substituted heteroaralkyl group. In one variation, R² is selected from the group consisting of hydrogen, an aralkyl group and a substituted heteroaralkyl group.

In one variation, R² is an aralkyl group where R² can be any one of the aralkyl groups noted for R¹ above, the same as if each and every aralkyl variation listed for R¹ is separately and individually listed for R².

In one variation, R² is a substituted heteroaralkyl group, where the alkyl moiety of the heteroaralkyl can be any alkyl or lower alkyl group, such as those listed above for R¹. In one variation, R² is a substituted heteroaralkyl where the heteroaryl group is substituted with 1 to 3 C₁-C₃ alkyl substituents (e.g., 6-methyl-3-pyridylethyl). In one variation, R² is a substituted heteroaralkyl group wherein the heteroaryl group is substituted with 1 to 3 methyl groups. In one variation, R² is a substituted heteroaralkyl group wherein the heteroaryl group is substituted with one lower alkyl substituent. In one variation, R² is a substituted heteroaralkyl group wherein the heteroaryl group is substituted with one C₁-C₃ alkyl substituent. In one variation, R² is a substituted heteroaralkyl group wherein the heteroaryl group is substituted with one or two methyl groups. In one variation, R² is a substituted heteroaralkyl group wherein the heteroaryl group is substituted with one methyl group.

In other variations, R² is any one of the substituted heteroaralkyl groups in the immediately preceding paragraph where the heteroaryl moiety of the heteroaralkyl group is a single ring heteroaryl group. In other variations, R² is any one of the substituted heteroaralkyl groups in the immediately preceding paragraph where the heteroaryl moiety of the heteroaralkyl group is a multiple condensed ring heteroaryl group. In other variations, R² is any one of the substituted heteroaralkyl groups in the immediately preceding paragraph where the heteroaralkyl moiety is a pyridyl group (Py).

In one variation, R² is 6-CH₃-3-Py-(CH₂)₂—. An example of a compound containing this moiety is dimebon.

In one variation, R³is hydrogen. In other variations, R³ is any one of the alkyl groups noted for R¹ above, the same as if each and every alkyl variation listed for R¹ is separately and individually listed for R³. In another variation, R³ is a halo group. In one variation, R³ is hydrogen or an alkyl group. In one variation, R³ is a halo or alkyl group. In one variation, R³is hydrogen or a halo group. In one variation, R³ is selected from the group consisting of hydrogen, alkyl and halo. In one variation, R³ is Br. In one variation, R³ is I. In one variation, R³ is F. In one variation, R³ is Cl.

In a particular variation, the hydrogenated pyrido[4,3-b]indole is 2,8-dimethyl-5-(2-(6-methyl-3-pyridyl)ethyl)-2,3,4,5-tetrahydro-1H-pyrido[4,3-b]indole or a pharmaceutically acceptable salt thereof.

The hydrogenated pyrido[4,3-b]indoles can be in the form of pharmaceutically acceptable salts thereof, which are readily known to those of skill in the art. The pharmaceutically acceptable salts include pharmaceutically acceptable acid salts. Examples of particular pharmaceutically acceptable salts include hydrochloride salts or dihydrochloride salts. In a particular variation, the hydrogenated pyrido[4,3-b]indole is a pharmaceutically acceptable salt of 2,8-dimethyl-5-(2-(6-methyl-3-pyridyl)ethyl)-2,3,4,5-tetrahydro-1H-pyrido[4,3-b]indole, such as 2,8-dimethyl-5-(2-(6-methyl-3-pyridyl)ethyl)-2,3,4,5-tetrahydro-1H-pyrido[4,3-b]indole dihydrochloride (dimebon).

Particular hydrogenated pyrido[4,3-b]indoles can also be described by the Formula (1) or by the Formula (2):

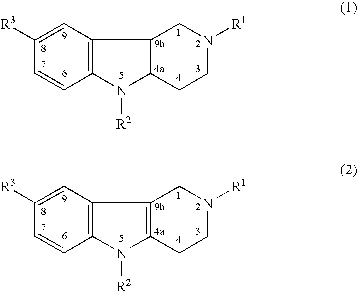

For compounds of a general Formula (1) or (2),
R¹ represents —CH₃, CH₃CH₂—, or PhCH₂-(benzyl);
R² is —H, PhCH₂—, or 6CH₃-3-Py-(CH2)₂—;
R³ is —H, —CH₃, or —Br,
   in any combination of the above substituents. All possible combinations of the substituents of Formula (1) and (2) are contemplated as specific and individual compounds the same as if each single and individual compound were listed by chemical name. Also contemplated are the compounds of Formula (1) or (2), with any deletion of one or more possible moieties from the substituent groups listed above: e.g., where R¹ represents —CH₃; R² is —H, PhCH₂—, or 6CH₃-3-Py-(CH₂)₂—; and R³ is —H, —CH₃, or —Br, or where R¹ represents —CH₃; R² is 6CH₃-3-Py-(CH₂)₂—; and R³ represents —H, —CH₃, or —Br.

The above and any compound herein may be in a form of salts with pharmaceutically acceptable acids and in a form of quatemized derivatives.

The compound may be Formula (1), where R¹ is —CH₃, R² is —H, and R³ is —CH₃. The compound may be Formula (2), where R¹ is represented by —CH₃, CH₃CH₂—, or PhCH₂—; R² is —H, PhCH₂—, or 6CH₃-3-Py-(CH₂)₂—; R³ is —H, —CH₃, or —Br. The compound may be Formula (2), where R¹ is CH₃CH₂— or PhCH₂—, R² is —H, and R³ is —H; or a compound, where R¹ is —CH₃, R² is PhCH₂—, R³ is —CH₃; or a compound, where R¹ is —CH₃, R² is 6-CH₃-3-Py-(CH₂)₂—, and R³ is —CH₃; or a compound, where R¹ is —CH₃, R² is —H, R³ is —H or —CH₃; or a compound, where R¹ is —CH₃, R² is —H, R³ is —Br.

Compounds known from literature which can be used in the methods disclosed herein include the following specific compounds:
1. cis(±) 2,8-dimethyl-2,3,4,4a,5,9b-hexahydro-1H-pyrido[4,3-b]indole and its dihydrochloride;
2. 2-ethyl-2,3,4,5-tetrahydro-1H-pyrido[4,3-b]indole;
3. 2-benzyl-2,3,4,5-tetrahydro-1H-pyrido[4,3-b]indole;
4. 2,8-dimethyl-5-benzyl-2,3,4,5-tetrahydro-1H-pyrido[4,3-b]indole and its dihydrochloride;
5. 2-methyl-5-(2-methyl-3-pyridyl)ethyl-2,3,4,5-tetrahydro-1H-pyrido[4,3-b]indole and its sesquisulfate;
6. 2, 8-dimethyl-5-(2-(6-methyl-3-pyridyl)ethyl)-2,3,4,5-tetrahydro-1H-pyrido[4,3-b]indole and its dihydrochloride (dimebon);
7. 2-methyl-2,3,4,5-tetrahydro-1H-pyrido[4,3-b]indole;
8. 2,8-dimethyl-2,3,4,5-tetrahydro-1H-pyrido[4,3-b]indole and its methyl iodide;
9. 2-methyl-8-bromo-2,3,4,5-tetrahydro-1H-pyrido[4,3-b]indole and its hydrochloride.

In one variation, the compound is of the Formula A or B and R¹ is selected from a lower alkyl or benzyl; R² is selected from a hydrogen, benzyl or 6-CH₃-3-Py-(CH₂)₂— and R³ is selected from hydrogen, lower alkyl or halo, or any pharmaceutically acceptable salt thereof. In another variation, R¹ is selected from —CH₃, CH₃CH₂—, or benzyl; R² is selected from —H, benzyl, or 6-CH₃-3-Py-(CH₂)₂—; and R³ is selected from —H, —CH₃ or —Br, or any pharmaceutically acceptable salt thereof. In another variation the compound is selected from the group consisting of: cis(±) 2,8-dimethyl-2,3,4,4a,5,9b-hexahydro-1H-pyrido[4,3-b]indole as a racemic mixture or in the substantially pure (+) or substantially pure (−) form; 2-ethyl-2,3,4,5-tetrahydro-1H-pyrido[4,3-b]indole; 2-benzyl-2,3,4,5-tetrahydro-1H-pyrido[4,3-b]indole; 2,8-dimethyl-5-benzyl-2,3,4,5-tetrahydro-1H-pyrido[4,3-b]indole; 2-methyl-5-(2-methyl-3-pyridyl)ethyl-2,3,4,5-tetrahydro-1H-pyrido[4,3-b]indole; 2,8-dimethyl-5-(2-(6-methyl-3-pyridyl)ethyl)-2,3,4,5-tetrahydro-1H-pyrido[4,3-b]indole; 2-methyl-2,3,4,5-tetrahydro-1H-pyrido[4,3-b]indole; 2,8-dimethyl-2,3,4,5-tetrahydro-1H-pyrido[4,3-b]indole; or 2-methyl-8-bromo-2,3,4,5-tetrahydro-1H-pyrido[4,3-b]indole or any pharmaceutically acceptable salt of any of the foregoing. In one variation, the compound is of the formula A or B wherein R¹ is —CH₃, R² is —H and R³ is —CH₃ or any pharmaceutically acceptable salt thereof. The compound may be of the Formula A or B where R¹ CH₃CH₂— or benzyl, R² is —H, and R³ is —CH₃ or any pharmaceutically acceptable salt thereof. The compound may be of the Formula A or B where R¹ is —CH₃, R² is benzyl, and R³ is —CH₃ or any pharmaceutically acceptable salt thereof. The compound may be of the Formula A or B where R¹ is —CH₃, R² is 6-CH₃-3-Py-(CH₂)₂—, and R³ is —H or any pharmaceutically acceptable salt thereof. The compound may be of the Formula A or B where R² is 6-CH₃-3-Py-(CH₂)₂— or any pharmaceutically acceptable salt thereof. The compound may be of the Formula A or B where R¹ is —CH₃, R² is —H, and R³ is —H or —CH₃ or any pharmaceutically acceptable salt, thereof. The compound may be of the Formula A or B where R¹ is —CH₃, R² is —H, and R³ is —Br, or any pharmaceutically acceptable salt thereof. The compound may be of the Formula A or B where R¹ is selected from a lower alkyl or aralkyl, R² is selected from a hydrogen, aralkyl or substituted heteroaralkyl and R³ is selected from hydrogen, lower alkyl or halo.

The compound for use herein may be 2,8-dimethyl-5-(2-(6-methyl-3-pyridyl)ethyl-2,3,4,5-tetrahydro-1H-pyrido[4,3-b]indole or any pharmaceutically acceptable salt thereof, such as an acid salt, a hydrochloride salt or a dihydrochloride salt thereof.

Any of the compounds disclosed herein having two stereocenters in the pyrido[4,3-b]indole ring structure (e.g., carbons 4a and 9b of compound (1)) includes compounds whose stereocenters are in a cis or a trans form. A composition may comprise such a compound in substantially pure form, such as a composition of substantially pure S,S or R,R or S,R or R,S compound. A composition of substantially pure compound means that the composition contains no more than 15% or no more than 10% or no more than 5% or no more than 3% or no more than 1% impurity of the compound in a different stereochemical form. For instance, a composition of substantially pure S,S compound means that the composition contains no more than 15% or no more than 10% or no more than 5% or no more than 3% or no more than 1% of the R,R or S,R or R,S form of the compound. A composition may contain the compound as mixtures of such stereoisomers, where the mixture may be enanteomers (e.g., S,S and R,R) or diastereomers (e.g., S,S and R,S or S,R) in equal or unequal amounts. A composition may contain the compound as a mixture of 2 or 3 or 4 such stereoisomers in any ratio of stereoisomers. Compounds disclosed herein having stereocenters other than in the pyrido[4,3-b]indole ring structure intends all stereochemical variations of such compounds, including but not limited to enanteomers and diastereomers in any ratio, and includes racemic and enantioenriched and other possible mixtures. Unless stereochemistry is explicitly indicated in a structure, the structure is intended to embrace all possible stereoisomers of the compound depicted.

Compounds listed above as compounds 1-9 from the literature are detailed in the following publications. Synthesis and studies on neuroleptic properties for cis(±) 2,8-dimethyl-2,3,4,4a,5,9b-hexahydro-1H-pyrido[4,3-b]indole and its dihydrochloride are reported, for instance, in the following publication: Yakhontov, L. N., Glushkov, R. G., Synthetic therapeutic drugs. A. G. Natradze, the editor, Moscow Medicina, 1983, p. 234-237. Synthesis of compounds 2, 8, and 9 above, and data on their properties as serotonin antagonists are reported in, for instance, in C. J. Cattanach, A. Cohen & B. H. Brown in J. Chem. Soc. (Ser.C) 1968, p. 1235-1243. Synthesis of the compound 3 above is reported, for instance, in the article N. P. Buu-Hoi, O. Roussel, P. Jacquignon, J. Chem. Soc., 1964, N 2, p. 708-711. N. F. Kucherova and N. K. Kochetkov (General chemistry (russ.), 1956, v. 26, p. 3149-3154) describe the synthesis of the compound 4 above. Synthesis of compounds 5 and 6 above is described in the article by A. N. Kost, M. A. Yurovskaya, T. V. Mel'nikova, in Chemistry of Heterocyclic Compounds, 1973, N 2, p. 207-212. The synthesis of the compound 7 above is described by U. Horlein in Chem. Ber., 1954, Bd. 87, hft 4, 463-p. 472. M. Yurovskaya and I. L. Rodionov in Chemistry of Heterocyclic Compounds (1981, N 8, p. 1072-1078) describe the synthesis of methyl iodide of the compound 8 above.

Methods, Formulations and Kits

As discussed in the examples, such as in Example 2, it was found that dimebon was effective in inhibiting mutant huntingtin-induced neurodegeneration of photoreceptor neurons in Drosophila eyes, which are reflective of neurodegerative changes in Drosophila brains. The Drosophila model is an established model for Huntington's disease. Thus, dimebon and the compounds described herein, such as compounds described by Formula (A), (1), (2) and (B), may be useful in treating, preventing, slowing the progression and/or delaying the onset and/or development of Huntington's disease.

The compounds described herein, such as dimebon or other compounds such as those described by the Formula (1) or (A) or (B) or by Formula (2), may be useful for their prophylactic effects or for their therapeutic application in medicine for delaying the onset and/or development of Huntington's disease and/or for treating Huntington's disease. For prophylactic use, beneficial or desired results includes results such as eliminating or reducing the risk, lessening the severity, or delaying the onset or outset of the disease, including biochemical, histologic and/or behavioral symptoms of the disease, its complications and intermediate pathological phenotypes presenting during development of the disease. For therapeutic use, beneficial or desired results includes clinical results such as inhibiting or suppressing the degeneration of neurons (such as neurons in the basal ganglia), improving cognition or reversing cognitive decline, decreasing one or more symptoms resulting from the disease (e.g., a biochemical, histologic, motor, cognitive and/or behavioral symptom) including its complications and intermediate pathological phenotypes presenting during development of the disease, increasing the quality of life of those suffering from the disease, decreasing the dose of other medications required to treat the disease, enhancing the effect of another medication, delaying the progression of the disease, and/or prolonging survival of patients. Exemplary symptoms that can be improved, eliminated, delayed, or prevented include one or more of the following: involuntary movements, cognition impairment or loss of cognitive function, chorea (involuntary writhing and spasming), clumsiness, impairment or loss of the ability to walk, impairment or loss of the ability to speak (e.g., exhibiting slurred speech), impairment or loss of the ability to swallow, impairment of intellectual speed, impairment of attention, impairment of short-term memory, change in personality, depression, irritability, emotional outburst, and apathy.

As used herein, “delaying” development of Huntington's disease includes deferring, hindering, slowing, retarding, stabilizing, and/or postponing development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of Huntington's disease is a method that reduces probability of disease development in a given time frame and/or reduces extent of the disease in a given time frame, when compared to not using the method. Such comparisons may be based on clinical studies, using a statistically significant number of subjects. Exemplary symptoms that can be delayed include one or more of the following: involuntary movements, cognition impairment or loss of cognitive function, chorea (involuntary writhing and spasming), clumsiness, impairment or loss of the ability to walk, impairment or loss of the ability to speak (e.g., exhibiting slurred speech), impairment or loss of the ability to swallow, impairment of intellectual speed, impairment of attention, impairment of short-term memory, change in personality, depression, irritability, emotional outburst, and apathy. Huntington's disease development can be detectable using standard clinical techniques (e.g., a standard neurological examination, patient interview, or more specialized testing). Development may also refer to disease progression that may be initially undetectable and includes occurrence, recurrence, and onset.

The present invention provides a variety of methods, such as those described in the “Brief Summary of the Invention” and elsewhere in this disclosure. The methods of the invention employ the compounds described herein. For example, in one embodiment, the present invention provides a method of treating Huntington's disease in a patient in need thereof comprising administering to the individual an effective amount of a hydrogenated pyrido[4,3-b]indole, such as dimebon, or pharmaceutically acceptable salt thereof. In one embodiment, the present invention provides a method of delaying the onset and/or development of Huntington's disease in an individual who is considered at risk for developing Huntington's disease, for example an individual whose one or more family members have had Huntington's disease comprising administering to the individual an effective amount of a hydrogenated pyrido[4,3-b]indole, such as dimebon, or pharmaceutically acceptable salt thereof. In one embodiment, the present invention provides a method of delaying the onset and/or development of Huntington's disease in an individual who is genetically predisposed to developing Huntington's disease comprising administering to the individual an effective amount of a hydrogenated pyrido[4,3-b]indole, such as dimebon, or pharmaceutically acceptable salt thereof. In one embodiment, the present invention provides a method of delaying the onset and/or development of Huntington's disease in an individual having a mutated or abnormal gene which codes for the mutant huntingtin protein but who has not been diagnosed with Huntington's disease comprising administering to the individual an effective amount of a hydrogenated pyrido[4,3-b]indole, such as dimebon, or pharmaceutically acceptable salt thereof. In one embodiment, the present invention provides a method of preventing Huntington's disease in an individual who is genetically predisposed to developing Huntington's disease or who has a mutated or abnormal gene which codes for the mutant huntingtin protein but who has not been diagnosed with Huntington's disease comprising administering to the individual an effective amount of a hydrogenated pyrido[4,3-b]indole, such as dimebon, or pharmaceutically acceptable salt thereof. In one embodiment, the present invention provides a method of preventing the onset and/or development of Huntington's disease in an individual who is not identified as genetically predisposed to developing Huntington's disease comprising administering to the individual an effective amount of a hydrogenated pyrido[4,3-b]indole, such as dimebon, or pharmaceutically acceptable salt thereof. In one embodiment, the present invention provides a method of decreasing the intensity or severity of the symptoms of Huntington's disease in an individual who is diagnosed with Huntington's disease comprising administering to the individual an effective amount of a hydrogenated pyrido[4,3-b]indole, such as dimebon, or pharmaceutically acceptable salt thereof.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the method comprises the manufacture of a medicament for use in any of the methods disclosed, e.g., treating and/or preventing and/or delaying the onset and/or development of Huntington's disease.

According to the present invention, methods of the present invention may comprise the administration to an individual of a pharmacological composition that contains an effective amount of hydrogenated pyrido[4,3-b]indoles described by the Formula (1) or by Formula (2) or any other hydrogenated pyrido[4,3-b]indoles described herein, such as those described in Formula (A) and (B), in dose of between about 0.1 and about 10 mg/kg of the body weight, at least once a day and during the period of time, which is required to achieve the therapeutic effect. In other variations, the daily dose (or other dosage frequency) of a hydrogenated pyrido[4,3-b]indole as described herein is between about 0.1 and about 8 mg/kg; or between about 0.1 to about 6 mg/kg; or between about 0.1 and about 4 mg/kg; or between about 0.1 and about 2 mg/kg; or between about 0.1 and about 1 mg/kg; or between about 0.5 and about 10 mg/kg; or between about 1 and about 10 mg/kg; or between about 2 and about 10 mg/kg; or between about 4 to about 10 mg/kg; or between about 6 to about 10 mg/kg; or between about 8 to about 10 mg/kg; or between about 0.1 and about 5 mg/kg; or between about 0.1 and about 4 mg/kg; or between about 0.5 and about 5 mg/kg; or between about 1 and about 5 mg/kg; or between about 1 and about 4 mg/kg; or between about 2 and about 4 mg/kg; or between about 1 and about 3 mg/kg; or between about 1.5 and about 3 mg/kg; or between about 2 and about 3 mg/kg; or between about 0.01 and about 10 mg/kg; or between about 0.01 and 4 mg/kg; or between about 0.01 mg/kg and 2 mg/kg; or between about 0.05 and 10 mg/kg; or between about 0.05 and 8 mg/kg; or between about 0.05 and 4 mg/kg; or between about 0.05 and 4 mg/kg; or between about 0.05 and about 3 mg/kg; or between about 10 kg to about 50 kg; or between about 10 to about 100 mg/kg or between about 10 to about 250 mg/kg; or between about 50 to about 100 mg/kg or between about 50 and 200 mg/kg; or between about 100 and about 200 mg/kg or between about 200 and about 500 mg/kg; or a dosage over about 100 mg/kg; or a dosage over about 500 mg/kg. In some embodiments, a daily dosage of dimebon is administered. The daily dosage for dimebon can be a 10 mg/kg dosage. An individual can receive a daily dosage of, for example, of about 60 mg/day or about 300 mg/day or from about 50 mg/day to about 500 mg/day or from about 10 mg/day to about 800 mg/day.

For use herein, unless clearly indicated otherwise, the compounds may be administered to the individual by any available dosage form. In one variation, the compound is administered to the individual as a conventional immediate release dosage form. In one variation, the compound is administered to the individual as a sustained release form or part of a sustained release system, such as a system capable of sustaining the rate of delivery of a compound to an individual for a desired duration, which may be an extended duration such as a duration that is longer than the time required for a corresponding immediate-release dosage form to release the same amount (e.g., by weight or by moles) of compound, and can be hours or days. A desired duration may be at least the drug elimination half life of the administered compound and may be about any of, e.g., at least about 6 hours or at least about 12 hours or at least about 24 hours or at least about 30 hours or at least about 48 hours or at least about 72 hours or at least about 96 hours or at least about 120 hours or at least about 144 or more hours, and can be at least about one week, at least about 2 weeks, at least about 3 weeks, at least about 4 weeks, at least about 8 weeks, or at least about 16 weeks or more.

The compound may be formulated for any available delivery route, whether immediate or sustained release, including an oral, mucosal (e.g., nasal, sublingual, vaginal, buccal or rectal), parenteral (e.g., intramuscular, subcutaneous or intravenous), topical or transdermal delivery form. A compound may be formulated with suitable carriers to provide delivery forms, which may be but are not required to be sustained release forms, that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein can be used in the preparation of a formulation, such as a pharmaceutical formulation, by combining the compound or compounds as an active ingredient with a pharmacologically acceptable carrier, which are known in the art, such as those mentioned above. Depending on the therapeutic form of the system (e.g., transdermal patch vs. oral tablet), the carrier may be in various forms. In addition, pharmaceutical preparations may contain preservatives, solubilizers, stabilizers, re-wetting agents, emulgators, sweeteners, dyes, adjusters, salts for the adjustment of osmotic pressure, buffers, coating agents or antioxidants. Preparations comprising the compound, such as dimebon, may also contain other substances which have valuable therapeutic properties. Therapeutic forms may be represented by a usual standard dose and may be prepared by a known pharmaceutical method. Suitable formulations can be found, e.g., in Remington 's Pharmaceutical Sciences, Mack Publishing Company, Philadelphia, Pa., 20^{th} ed. (2000), which is incorporated herein by reference.

Compounds described by Formula (1) or by Formula (2) or compounds described by Formula (A) or (B), such as dimebon, may be administered to individuals in a form of generally accepted oral compositions, such as tablets, coated tablets, gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical preparations may contain preservatives, solubilizers, stabilizers, re-wetting agents, emulgators, sweeteners, dyes, adjusters, salts for the adjustment of osmotic pressure, buffers, coating agents or antioxidants. Preparations comprising the compound, such as dimebon, may also contain other substances which have valuable therapeutic properties. Therapeutic forms may be represented by a usual standard dose and may be prepared by a known pharmaceutical method. Suitable formulations can be found, e.g., in Remington 's Pharmaceutical Sciences, Mace Publishing Company, Philadelphia, Pa., 20^{th} ed. (2000), which is incorporated herein by reference. Any of the compounds described herein can be formulated in a tablet in any dosage form described, for example, dimebon or a pharmaceutically acceptable salt thereof can be formulated as a 10 mg tablet. Any of the compounds described herein can be formulated in any dosage as a sustained release formulation. Sustained release formulations can be prepared in various delivery systems, including but not limited to oral dosing forms, IM depot forms, and forms amenable to CNS delivery or implantation. The invention also provides for a sustained release device, for example a transdermal patch or an implantable device comprising as the active ingredient any one of the compounds described herein in any total amount such that the individual receives an effective amount of compound during the sustained release period. The technical result that may be achieved after the application of the present invention may be a treatment of Huntington's disease, a delayed onset and/or development of Huntington's disease, slowing the progression of Huntington's disease or prophylactically protecting the individual from ever developing Huntington's disease.

The amount of compound such as dimebon in a delivery form may be any effective amount, which may be from about 10 ng to about 1,500 mg or more. In one variation, a delivery form, such as a sustained release system, comprises less than about 30 mg of compound. In one variation, a delivery form, such as a single sustained release system capable of multi-day administration, comprises an amount of compound such that the daily dose of compound is less than about 30 mg of compound.

A treatment regimen involving a dosage form of compound, whether immediate release or a sustained release system, may involve administering the compound to the individual in dose of between about 0.1 and about 10 mg/kg of body weight, at least once a day and during the period of time required to achieve the therapeutic effect. In other variations, the daily dose (or other dosage frequency) of a hydrogenated pyrido[4,3-b]indole as described herein is between about 0.1 and about 8 mg/kg; or between about 0.1 to about 6 mg/kg; or between about 0.1 and about 4 mg/kg; or between about 0.1 and about 2 mg/kg; or between about 0.1 and about 1 mg/kg; or between about 0.5 and about 10 mg/kg; or between about 1 and about 10 mg/kg; or between about 2 and about 10 mg/kg; or between about 4 to about 10 mg/kg; or between about 6 to about 10 mg/kg; or between about 8 to about 10 mg/kg; or between about 0.1 and about 5 mg/kg; or between about 0.1 and about 4 mg/kg; or between about 0.5 and about 5 mg/kg; or between about 1 and about 5 mg/kg; or between about 1 and about 4 mg/kg; or between about 2 and about 4 mg/kg; or between about 1 and about 3 mg/kg; or between about 1.5 and about 3 mg/kg; or between about 2 and about 3 mg/kg; or between about 0.01 and about 10 mg/kg; or between about 0.01 and 4 mg/kg; or between about 0.01 mg/kg and 2 mg/kg; or between about 0.05 and 10 mg/kg; or between about 0.05 and 8 mg/kg; or between about 0.05 and 4 mg/kg; or between about 0.05 and 4 mg/kg; or between about 0.05 and about 3 mg/kg; or between about 10 kg to about 50 kg; or between about 10 to about 100 mg/kg or between about 10 to about 250 mg/kg; or between about 50 to about 100 mg/kg or between about 50 and 200 mg/kg; or between about 100 and about 200 mg/kg or between about 200 and about 500 mg/kg; or a dosage over about 100 mg/kg; or a dosage over about 500 mg/kg. In some embodiments, a daily dosage of dimebon is administered, such as a daily dosage that is less than about 0.1 mg/kg, which may include but is not limited to, a daily dosage of about 0.05 mg/kg. In one variation, the dosage amount is a human dosage that is extracted from and correlates to a 10 μM to 1 mM dosage solution administered to Drosophila.

The compound, such as dimebon, may be administered to an individual in accordance with an effective dosing regimen for a desired period of time or duration, such as at least about one month, at least about 2 months, at least about 3 months, at least about 6 months, or at least about 12 months or longer. In one variation, the compound is administered on a daily or intermittent schedule for the duration of the individual's life. The compound can be administered to an individual continuously (for example, at least once daily) over a period of time. In some embodiments, the compound is administered to an individual for at least about three months. In some embodiments, the compound is administered to an individual for at least about six months. In some embodiments, the compound is administered to an individual for at least about twelve months. In some embodiments, the compound is administered to an individual for the duration of the individual's life. In some embodiments, the compound can be administered as an oral or depot drug given to sustain therapeutic benefits for an individual's lifetime. The compound can be administered as a daily oral administration of the compound or as pharmacokinetics allows, a lesser dosing such as once weekly dosing.

The dosing frequency can be about a once weekly dosing. The dosing frequency can be about a once daily dosing. The dosing frequency can be more than about once weekly dosing. The dosing frequency can be less than three times a day dosing. The dosing frequency can be less than about three times a day dosing. The dosing frequency can be about three times a week dosing. The dosing frequency can be about a four times a week dosing. The dosing frequency can be about a two times a week dosing. The dosing frequency can be more than about once weekly dosing but less than about daily dosing. The dosing frequency can be about a once monthly dosing. The dosing frequency can be about a twice weekly dosing. The dosing frequency can be more than about once monthly dosing but less than about once weekly dosing. The dosing frequency can be intermittent (e.g., once daily dosing for 7 days followed by no doses for 7 days, repeated for any 14 day time period, such as about 2 months, about 4 months, about 6 months or more). The dosing frequency can be continuous (e.g., once weekly dosing for continuous weeks). Any of the dosing frequencies can employ any of the compounds described herein together with any of the dosages described herein, for example, the dosing frequency can be a once daily dosage of less than 0.1 mg/kg or less than about 0.05 mg/kg of dimebon.

The compound, such as dimebon, or pharmacological composition comprising the compound may be administered for a sustained period, such as at least about one month, at least about 2 months, at least about 3 months, at least about 6 months, or at least about 12 months or longer. The compound may be administered for the duration of the individual's life.

Other dosing schedules of the compound, such as dimebon, or pharmacological composition may also be followed. For example, the frequency of the administration may vary. The dosing frequency can be a once weekly dosing. The dosing frequency can be a once daily dosing. The dosing frequency can be more than once weekly dosing. The dosing frequency can be more than once daily dosing, such as any one of 2, 3, 4, 5, or more than 5 daily doses. The dosing frequency can be 3 times a day. The dosing frequency can be three times a week dosing. The dosing frequency can be a four times a week dosing. The dosing frequency can be a two times a week dosing. The dosing frequency can be more than once weekly dosing but less than daily dosing. The dosing frequency can be about a once monthly dosing. The dosing frequency can be about a twice weekly dosing. The dosing frequency can be more than about once monthly dosing but less than about one weekly dosing. The dosing frequency can intermittent (e.g., one daily dosing for 7 days followed by no doses for 7 days, repeated for any 14 day time period, such as about 2 months, about 4 months, about 6 months or more). The dosing frequency can be continuous (e.g., one weekly dosing for continuous weeks). Any of the dosing frequencies for any of the compounds or pharmacological compositions disclosed herein, such as dimebon, can be used with any dosage amount, for example, any of the dosing frequencies can employ a 10 mg/kg or 20 mg/kg dosage amount or any other dosage amount disclosed herein. Any of the dosing frequencies can employ any of the compounds described herein together with any of the dosages described herein, for example, the dosing frequency can be a three times daily 10 mg/kg dose of dimebon.

The invention further provides kits for carrying out the methods of the invention, which comprises one or more compounds described herein or a pharmacological composition comprising a compound described herein. The kits may employ any of the compounds disclosed herein. In one variation, the kit employs dimebon or a pharmaceutically acceptable salt thereof, such as the dihydrochloride salt. The kits may be used for any one or more of the uses described herein, and, accordingly, may contain instructions for any one or more of the following uses: treating Huntington's disease, preventing Huntington's disease, and/or delaying the onset and/or development of Huntington's disease.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The invention also provides compositions (including pharmacological compositions) as described herein for the use in treating Huntington's disease, preventing Huntington's disease, delaying the onset and/or development of Huntington's disease and other methods described herein.

The following Examples are provided to illustrate but not limit the invention.

### EXAMPLES

In the examples below, data was typically evaluated using ANOVA analysis followed by Dunnett's post-hoc test. ANOVA (analysis of variance) compares all datasets together indicating whether there is a significant difference between the datasets. If ANOVA showed a significant difference, a post-hoc multiple comparison test (Dunnett's test) may be applied that compares datasets in a pair-wise manner.

### Example 1

### Determination of Toxicity Properties of Dimebon

Dimebon, 2,8-dimethyl-5-(2-(6-methyl-3-pyridyl)-ethyl)-2,3,4,5-tetrahydro-1H-pyrido[4,3-b]indole dihydrochloride, was used as a representative compound of hydrogenated pyrido[4,3-b]indoles. 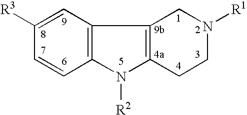
×2 HCl
where R¹ and R³ are methyls, and
R² is 2-(6-methyl-3-pyridyl)-ethyl

Dimebon was evaluated for toxicity levels in wildtype Drosophila fruit flies. Dimebon was administered daily at doses ranging from 10 μM to 1 mM to explore its toxicity. An untreated control group was also studied in this experiment. The concentrations given were concentrations of dimebon in the food that animals drink/eat ad libitum. The food consisted of cornmeal (61.2 g), dextrose (129.4. g), yeast (32.4 g) and agar (9.3 g) in 1 liter of water. Concentrated compounds were added to melted agar food cooled to just above the point of setting (about 40° C.), mixed and dispensed.

About 500 wild type Drosophila eggs were collected on grape juice plates for 20 hours, washed with distilled water and transferred 100 per vial to grow at 25 degrees C. The adult progeny were scored after eclosing (emerging from the pupal case) beginning 10 days later to assess developmental delay and toxicity. The criteria used for toxicity were the number (%) of animals that eclose and the time of the eclosing. For example, fewer animals may emerge from the pupal case if a drug is toxic or the same number of animals may eclose but more slowly than the untreated control group.

The toxicity test employed five concentrations (10 μM, 30 μM, 100 μM, 300 μM, 1 mM) plus an untreated control and was repeated with five sets of flies. The toxicity results indicate that dimebon is generally well tolerated over the range of 10 to 300 μM in the food. At 1 mM, there was approximately a 20% decrease in eclosion observed, indicating toxicity at this food concentration. ANOVA analysis indicated a significant difference between datasets (p=0.0031), with post-hoc testing indicating a significant difference between the control and 1 mM datasets (p<0.01).

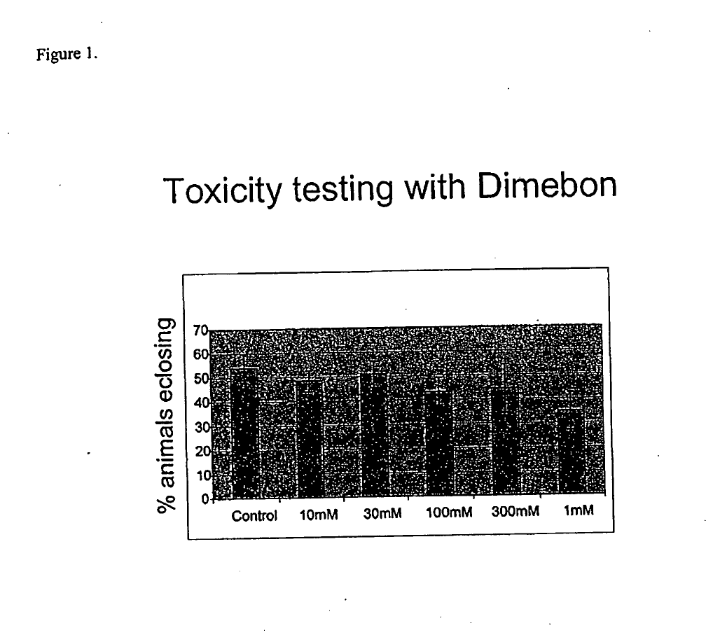

As illustrated in FIG. 1, dimebon caused no significant toxicity until a dose of 1 mM was reached, at which point there was a decrease in the % of animals eclosing and the timing of emergence was slowed by approximately 1 day.

### Example 2

### Determination of Dimebon's Ability to Inhibit Huntingtin-Induced Neurodegeneration of Photoreceptor Neurons in Drosophila Eyes

The gene responsible for Huntington's disease was discovered in 1993. This has allowed scientists to develop transgenic animal models of Huntington's disease. For instance, transgenic mouse, fly and worm models engineered to express the mutant gene causing Huntington's disease have greatly facilitated the discovery and elucidation of pathogenic mechanisms. In rodents and Drosophila fruit flies, the insertion of the huntingtin gene into the genomes of these animals has been shown to induce many of the pathological and clinical signs of Huntington's disease seen in humans and therefore the study of these transgenic animals is useful to assess the pharmacological activities of potential Huntington's disease therapeutic agents prior to testing them in humans.

The expression of mutant huntintin protein in Drosophila fruit flies results in a fly phenotype that exhibits some of the features of human Huntington's disease. First, the presumed etiologic agent in Huntington's disease (mutant huntingtin protein) is encoded by a repeated triplet of nucleotides (CAG) which are called polyglutamine or polyQ repeats. In humans, the severity of Huntington's disease is correlated with the length of polyQ repeats. The same polyQ length dependency is seen in Drosophila. Secondly, no neurodegeneration is seen at early ages (early larval stages) in flies expressing the mutant huntingtin protein, although at later life stages (mature larval, pupal and aging adult stages), flies do develop the disease, similarly to humans, who generally manifest the first signs and symptoms of Huntington's disease starting in the fourth and fifth decades of life. Third, the neurodegeneration seen in flies expressing the mutant huntingtin gene is progressive, as it is in human patients with Huntington's disease. Fourth, the neuropathology in huntingtin-expressing flies leads to a loss of motor function as it does in similarly afflicted human patients. Last, flies expressing the mutant huntingtin protein die an early death, as do patients with Huntington's disease. For these reasons, compounds which show a neuroprotective effect in the Drosophila model of Huntington's disease are expected to be the most likely compounds to have a beneficial effect in humans.

Dimebon, 2,8-dimethyl-5-(2-(6-methyl-3-pyridyl)-ethyl)-2,3,4,5-tetrahydro-1H-pyrido(4,3-b)indol dihydrochloride, was used as a representative compound of [4,3-b]indoles. 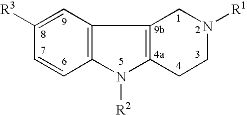
×2 HCl
where R¹ and R³ are methyls, and
R² is 2-(6-methyl-3-pyridyl)-ethyl

Dimebon was administered to one group of transgenic Drosophila engineered to express the mutant huntingtin protein in all their neurons. This was accomplished by cloning a foreign gene into transposable p-element DNA vectors under control of a yeast upstream activator sequence that is activated by the yeast GAL4 transcription factor. These promoter fusions were injected into fly embryos to produce transgenic animals. The foreign gene is silent until crossed to another transgenic strain of flies expressing the GAL4 gene in a tissue specific manner. The Elav>Gal4 which expresses the transgene in all neurons from birth until death was used in the experiments described.

For compound testing, 20-30 Httex1pQ93 virgins were mated to elav>Gal4 males and eggs were collected for about 20 hours at 25° C. and dispensed into vials (expected about 70% lethality from Htt effects). Upon eclosion, at least 80, 0-8 hour old flies were harvested and place on drug-containing food (20 eclosed adults per vial) and scored when 7 days old. Inhibitor-containing food was prepared just before tester flies began to emerge.

The two types of transgenic animals were crossed in order to collect enough closely matched aged controls to study. The crossed aged-matched adults (about 20 per dosing group) were placed on drug-containing food for 7 days. Animals were transferred to fresh food daily to minimize any effects caused by instability of the compounds. Survival was scored daily. The average number of photoreceptors at day zero was determined by scoring 7-10 of the newly eclosed tester siblings within six hours of eclosing. This established the baseline of degeneration at the time of exposure to drug. At day 7, animals were sacrificed and the number of photoreceptor neurons surviving was counted. Scoring was by the pseudopupil method where individual functioning photoreceptors were revealed by light focused on the back of the head and visualized as focused points of light under a compound microscope focused at the photoreceptor level of the eye. For pseudopupil analysis, flies were decapitated and the heads are mounted in a drop of nail polish on a microscopic slide. The head was then covered with immersion oil and light is projected through the eye of the fly using a Nikon EFD-3/Optiphot-2 compound microscope with a 50× oil objective. Dimebon was found to protect photoreceptors in a dose-dependant manner.

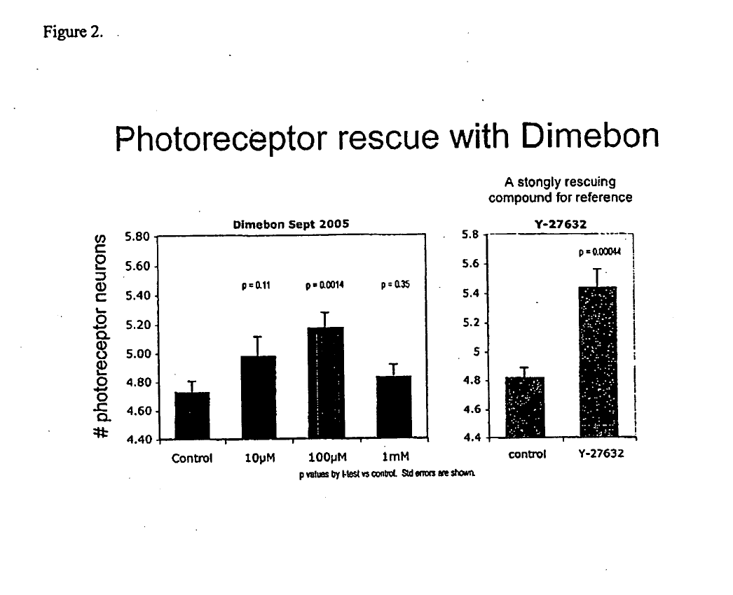

As shown in FIG. 2, when tested for its ability to inhibit mutant huntingtin-induced neurodegeneration of photoreceptor neurons in Drosophila eyes (which are reflective of neurodegenerative changes in fly brains), dimebon at a dose of 100 μM caused a statistically significant (p=0.0014) rescue of neurons compared to the untreated controls. The magnitude of effect seen is comparable to a historical positive control, Y-27632, a small molecule rho kinase inhibitor considered to be a strongly rescuing reference compound. A dose-dependent rescue of fly neurons was observed with dimebon, with a lesser but still apparent rescue of neurons observed at the 10 μM dose compared to the 100 μM dose. The 1 mM dimebon dose (established in the previous toxicity study to be a somewhat toxic dose) still appeared to cause neuronal rescue, but to a lesser extent than the 100 μM or 10 μM dimebon doses.

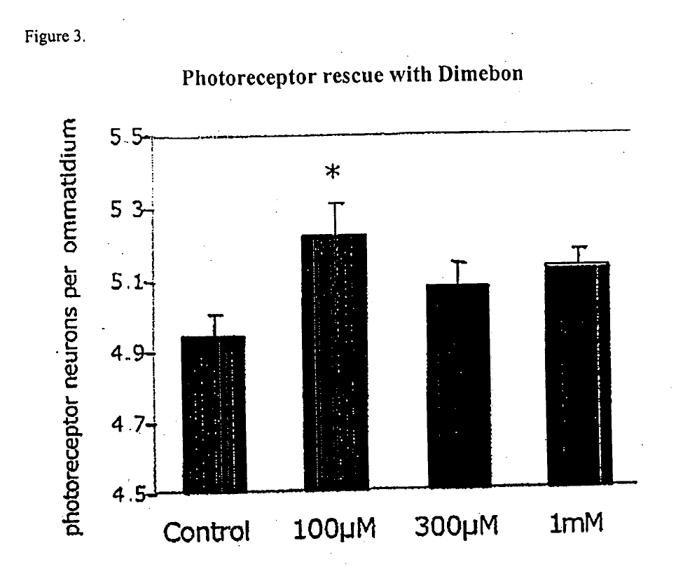

Animals were retested for suppression of photoreceptor neuron degeneration at concentrations around 100 μM, as shown in FIG. 3. The first retest examined the response to higher concentrations comparing rescue with 100, 300 or 1,000 μM dimebon to zero drug controls. Again, significant improvement was achieved at 100 μM with no statistically demonstrable improvement at higher doses. ANOVA tests revealed a significant difference between datasets P=0.0465 with a significant difference between the control and 100 μM datasets using Dunnett's multiple comparison test (P<0.05(*)).

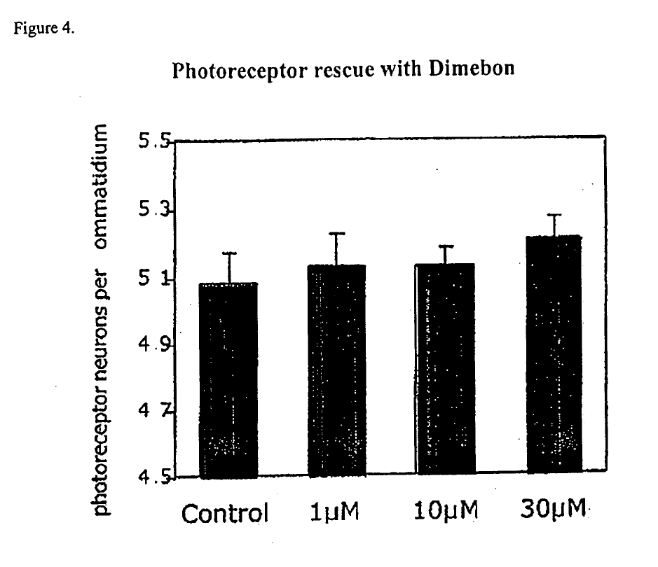

To determine the lowest effective dose in this study, animals were tested at lower concentrations using 1, 10 and 30 μM dimebon, as shown in FIG. 4. When more than five concentrations of drug were tested, the test was split into multiple days. This allowed time for the pseudopupil analysis. Since a difference was noticed between Elav>Gal4;UAS>HttQ93 adult flies that emerged on different days, No Drug controls were set up for each day. For this reason, the No Drug controls for the High and Low Dimebon concentration efficacy test are different from each other (e.g., see FIGS. 3 and 4). To analyze the data, the non-treated adults were compared to the drug treated adults that emerged on the same day. The previously measured optimum was 100 μM. As this was an initial test, no statistically significant improvement (P=0.7837 by ANOVA) was demonstrated at the lower concentrations despite the observed slight tendency for improvement as concentrations were increased.

### Example 3

### Determination of Dimebon's Effect on Motor Ability in a Drosophila Model

The motor function of Drosophila obtained as described in the examples above was assessed by exploiting the strong negative geotropism of flies to climb upwards when they are tapped to the bottom of a vial. See, Le Bourg and Lint (1992) Hypergravity and aging in Drosophila melanogaster. 4. Climing activity. Gerontology 38, 59-64. Animals were placed in a graduated vessel (e.g., a measuring cylinder). The distance climbed in 10 seconds was measured for each animal over 3 trials with a 5 minute rest period. In a separate experiment using tall thin plastic tubes rather than glass vials, the distance climbed in 30 seconds was also measured. Animals were scored for outcome without knowledge of treatment group.

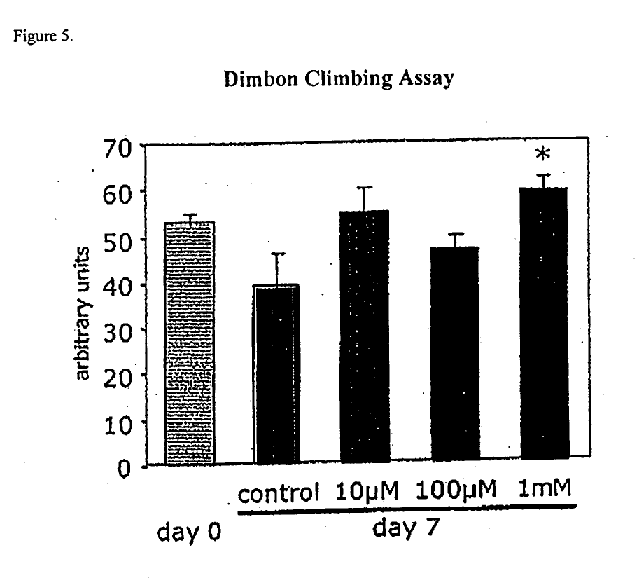

Flies were tested for functional rescue using a behavior assay (climbing assay) where the distance climbed was measured. Flies are negatively geotropic and hence immediately climb up the wall of a container if tapped down to the bottom. In this assay, climbing was scored blind and each animal was given 3 trials that were then averaged. The climbing of 7 day old animals reared on 0, 10, 100 or 1,000 μM containing food was compared as was the climbing of animals on the day of eclosion. Two trials were performed. In the first, the ability to climb in large glass vials was monitored over 10 seconds. ANOVA testing of the climbing results indicated a significant difference between datasets (P=0.0416) with a significant difference between the control and 1000 μM (P<0.05), as shown in FIG. 5.

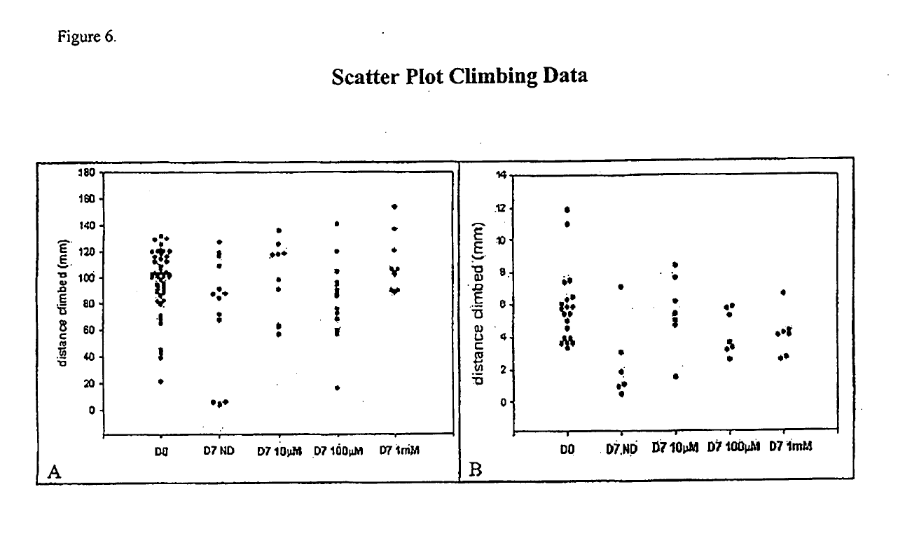

The second trial was similar to the first except that animals were tested in tall thin plastic tubes for climbing over 30 seconds. In this experiment, there was no statistically demonstrable difference between treated and control groups. The analysis of behavioral responses exhibited a high degree of variance. The data from the two independent climbing trials are plotted as individual data points in FIG. 6A and 6B. A higher statistical power (e.g., 0.8) given the observed standard deviations and differences of the means may be achieved by doubling the size of the experiment for the highest concentration and increasing about 10 fold for the lower concentrations.

Thus, one trial assessing climbing behavior over 10 seconds in glass tubes exhibited a statistically significant improvement in climbing with 1 mM dimebon. No statistically demonstrable difference between treated and control groups was observed in a second trial assessing climbing behavior over 30 seconds in a plastic tube. However, there may be improvement in motor function upon treatment with dimebon. For instance, larger scale experiments with a higher statistical power may be necessary to determine this conclusively. However, the absence of robust improvement in climbing behavior does not rule out a potential efficacious use of the compounds disclosed, such as dimebon, in the treatment of Huntington's disease.

### Example 4

### Use of Human Clinical Trials to Determine Ability of Compounds of the Invention to Treat, Prevent and/or Delay the Onset and/or the Development of HD

If desired, any of the hydrogenated pyrido[4,3-b]indoles described herein (e.g., dimebon) can also be tested in humans to determine the ability of the compound to treat, prevent and/or delay the onset and/or the development of Huntington's disease. Standard methods can be used for these clinical trials. In one exemplary method, subjects with Huntington's disease are enrolled in a tolerability, pharmacokinetics and pharmacodynamics phase I study of a hydrogenated pyrido[4,3-b]indole using standard protocols. Then a phase II, double-blind randomized controlled trial is performed to determine the efficacy of the hydrogenated pyrido[4,3-b]indole using standard protocols.

Experimental Summary

Dimebon appears to exhibit minimal toxicity and significant suppression of neuronal degeneration at 100 μM. The presented results suggest that dimebon statistically reliably inhibits mutant huntingtin-induced neurodegeneration of neurons in Drosophila eyes. Results in the described Drosophila model historically have correlated very well with transgenic mouse models for Huntington's disease. The close resemblance of the Drosophila model to the human Huntington's disease condition is described in J. L. Marsh et al, “Fly models of Huntington's Disease”, Human Molecular Genetics, 2003, vol 12, review issue 2, R187-R193. Thus, dimebon is believed to be a promising new agent for use in medicine to treat, prevent, slow the progression or delay the onset and/or development of Huntington's disease. All of the above suggest that dimebon and the class of compounds disclosed herein are promising effective agents for the treating, preventing, slowing the progression of or delaying the onset and/or development of Huntington's disease.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

All references, including patents, patent applications, and non-patent publications are hereby incorporated by reference herein in their entirety.

1. (canceled)

2. A method selected from the group consisting of:
(a) a method of treating Huntington's disease in an individual in need thereof;
(b) a method of slowing the progression of Huntington's disease in an individual who has a mutated or abnormal gene which codes for the mutant huntingtin protein or who expresses the mutant huntingtin protein; and
(c) a method of preventing or delaying development of Huntington's disease in an individual who is at risk of developing Huntington's disease;
the method comprising administering to an individual an effective amount of a hydrogenated pyrido[4,3-b]indole or pharmaceutically acceptable salt thereof.

3. The method of claim 2, wherein the hydrogenated pyrido[4,3-b]indole is a tetrahydro pyrido[4,3-b]indole.

4. The method of claim 2, wherein the hydrogenated pyrido[4,3-b]indole is a hexahydro pyrido[4,3-b]indole.

5. The method of claim 2, wherein the hydrogenated pyrido[4,3-b]indole is of the formula: 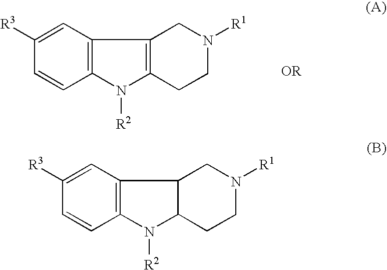
wherein:
R¹ is selected from a lower alkyl or aralkyl
R² is selected from a hydrogen, aralkyl or substituted heteroaralkyl
R³ is selected from hydrogen, lower alkyl or halo.

6. The method of claim 5, wherein aralkyl is PhCH₂— and substituted heteroaralkyl is 6-CH₃-3-Py-(CH₂)₂—.

7. The method of claim 5, wherein
R¹ is selected from CH₃—, CH₃CH₂—, or PhCH₂—
R² is selected from H—, PhCH₂—, or 6-CH₃-3-Py-(CH₂)₂—
R³ is selected from H—, CH₃— or Br—.

8. The method of claim 2, wherein the hydrogenated pyrido[4,3-b]indole is selected from the group consisting of:
cis(±)2,8-dimethyl-2,3,4,4a,5,9b-hexahydro-1H-pyrido[4,3-b]indole;
2-ethyl-2,3,4,5-tetrahydro-1H-pyrido[4,3-b]indole;
2-benzyl-2,3,4,5-tetrahydro-1H-pyrido[4,3-b]indole;
2,8-dimethyl-5-benzyl-2,3,4,5-tetrahydro-1H-pyrido[4,3-b]indole;
2-methyl-5-(2-methyl-3-pyridyl)ethyl-2,3,4,5-tetrahydro-1H-pyrido[4,3-b]indole;
2,8-dimethyl-5-(2-(6-methyl-3-pyridyl)ethyl)-2,3,4,5-tetrahydro-1H-pyrido[4,3-b]indole;
2-methyl-2,3,4,5-tetrahydro-1H-pyrido[4,3-b]indole;
2,8-dimethyl-2,3,4,5-tetrahydro-1H-pyrido[4,3-b]indole;
2-methyl-8-bromo-2,3,4,5-tetrahydro-1H-pyrido[4,3-b]indole.

9. The method of claim 8, wherein the hydrogenated pyrido[4,3-b]indole is 2,8-dimethyl-5-(2-(6-methyl-3-pyridyl)ethyl)-2,3,4,5-tetrahydro-1H-pyrido[4,3-b]indole.

10. The method of claim 8, wherein the pharmaceutically acceptable salt is a pharmaceutically acceptable acid salt.

11. The method of claim 10, wherein the pharmaceutically acceptable salt is a hydrochloride acid salt.

12. The method of claim 2, wherein the hydrogenated pyrido[4,3-b]indole is 2,8-dimethyl-5-(2-(6-methyl-3-pyridyl)ethyl)-2,3,4,5-tetrahydro-1H-pyrido[4,3-b]indole dihydrochloride.

13. The method of claim 7, wherein R¹ is CH₃—, R² is H and R³ is CH₃—.

14. The method of claim 7, wherein R¹ CH₃CH₂— or PhCH₂—, R² is H—, and R³ is CH₃—.

15. The method of claim 7, wherein R¹ is CH₃—, R² is PhCH₂—, and R³ is CH₃—.

16. The method of claim 7, wherein R¹ is CH₃—, R² is 6-CH₃-3-Py-(CH₂)₂—, and R³ is H—.

17. The method of claim 7, where R² is 6-CH₃-3-Py-(CH₂)₂—.

18. The method of claim 7, wherein R¹ is CH₃—, R² is H—, and R³ is H— or CH₃—.

19. The method of claim 7, where R¹ is CH₃—, R² is H—, and R³ is Br—.

20. A kit comprising: (a) a hydrogenated pyrido[4,3-b]indole or pharmaceutically acceptable salt thereof and (b) instructions for use of in treating, preventing, slowing the progression or delaying the onset and/or development of Huntington's disease.

21. The kit of claim 20, wherein the hydrogenated pyrido[4,3-b]indole is 2,8-dimethyl-5-(2-(6-methyl-3-pyridyl)ethyl)-2,3,4,5-tetrahydro-1H-pyrido[4,3-b]indole dihydrochloride.
